(19) United States
(12) Patent Application Publication
(10) Pub. No.: US 2003/0170181 A1
(43) Pub. Date: Sep. 11, 2003

(54) Method for preventing abuse of methylphenidate

(76) Inventor: Kamal K. Midha, Hamilton (BM)

Correspondence Address: REED & EBERLE LLP, 800 MENLO AVENUE, SUITE 210, MENLO PARK, CA, 94025, US

(21) Appl. No.: 10/321,778

(22) Filed: Dec. 16, 2002

Related U.S. Application Data
(63) Continuation-in-part of application No. 09/992,353, filed on Nov. 13, 2001, now Pat. No. 6,555,136.
Application No. 09/992,353 is a continuation of application No. 09/544,732, filed on Apr. 6, 2000, now Pat. No. 6,340,476.
(60) Provisional application No. 60/127,984, filed on Apr. 6, 1999.

(51) Int. Cl. A61K 49/00; A61K 35/78; A61K 31/445

(52) U.S. Cl. 424/10.4; 424/760; 514/317

(57) ABSTRACT

A method is provided for treating a patient with a methylphenidate-responsive condition that is at a risk of abusing or becoming addicted to methylphenidate. The patient is first evaluated for an elevated risk of drug abuse or addiction through psychological evaluations and then treated with a methylphenidate product that includes an emesis-inducing agent that is inert when ingested orally and only produces emesis when snorted or taken intravenously or a topical analgesic that is inert when ingested orally and only produces irritation when snorted or taken intravenously. The method includes delivering the methylphenidate in a pulsatile delivery system such that the emesis-inducing agent or the topical analgesic is included in one of the pulsatile dosages.

### CROSS-REFERENCE TO RELATED APPLICATIONS

This application is a continuation-in-part of U.S. patent application Ser. No. 09/992,353, filed Nov. 13, 2001, which is a continuation of U.S. patent application Ser. No. 09/544,732, filed Apr. 6, 2000, and issued as U.S. Pat. No. 6,340,476 on Jan. 22, 2002, which claimed priority under 35 U.S.C. §119(e)(1) to U.S. Provisional Patent Application Serial No. 60/127,984, filed Apr. 6, 1999. The disclosures of the aforementioned patent applications are incorporated by reference herein.

### TECHNICAL FIELD

The present invention relates generally to the fields of drug delivery, pharmacology, and medicine. More specifically, the invention relates to a method of reducing the likelihood of abuse of methylphenidate by administering a methylphenidate product that is formulated to prevent abuse of the drug. The methylphenidate product may be administered to the patient via a pulsatile drug release system.

### BACKGROUND

Methylphenidate hydrochloride (HCl), the hydrochloride salt of α-phenyl-2-piperidine-acetic acid methyl ester (available commercially as Ritalin®), is a central nervous system stimulant that is used in the treatment of Attention Deficit Disorder (“ADD”), a commonly diagnosed nervous system illness in children that is characterized by both distractibility and impulsivity. Methylphenidate HCl is also used to treat a related disorder, Attention Deficit Hyperactivity Disorder (“ADHD”), in which symptoms of hyperactivity are present along with the symptoms of ADD. The drug is additionally used in the symptomatic treatment of narcolepsy, chronic fatigue syndrome, depression, bulimia, and the cognitive decline associated with Acquired Immunodeficiency Syndrome (“AIDS”) or AIDS-related conditions, as well as for mood elevation, particularly in terminally ill patients with diseases such as cancer. Methylphenidate exists as four distinct isomers, as follows:

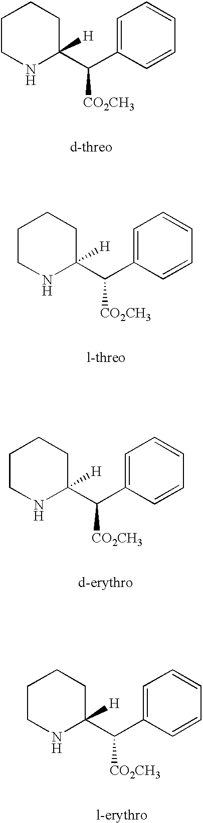

The drug as used in therapy is a racemic mixture of the d- and l-threo enantiomers, which have been acknowledged as more active than the erythro pair. The racemic d,l-threo methylphenidate (hereinafter referred to as “methylphenidate,” “d,l-MPH,” or “MPH”) is a short-acting stimulant with a duration of action of one to four hours and a pharmacokinetic elimination half-life of two to three hours. Maximum drug concentration after oral administration occurs at about two hours, at which time, the methylphenidate has been absorbed from the gastrointestinal tract and has passed into the systemic circulation including the brain. It is believed that once in the brain, methylphenidate influences neurotransmitters by inhibiting the uptake of dopamine in the striatum. Kimko, H. C., et al., “Pharmacokinetics and Clinical Effectiveness of Methylphenidate,” Clinical Pharmacokinetics 37(b):457-470 (December, 1999). The therapeutic dosage for methylphenidate is typically very low with tablets being dispensed in 5 mg, 10 mg, and 20 mg dosages, concentrations often referred to as “microdoses.” Side effects of the drug in its therapeutic dosages include anorexia, weight loss, insomnia, dizziness, and dysphoria. See, e.g., U.S. Pat. No. 6,410,746 to Davies.

Methylphenidate has a short half-life and a high potential for tolerance because it loses clinical efficacy when constant blood levels are maintained. See, U.S. Pat. No. 6,340,476 to Midha et al. Studies on the activity of methylphenidate have shown that the d-threo enantiomer is the active material in the racemic drug and that the l-threo enantiomer contributes minimally to the ADHD activity of the drug. See, e.g., U.S. Pat. No. 5,773,478 to Richards, et al. Further, while the l-threo enantiomer is rapidly and stereoselectively metabolized upon oral administration of the racemic drug, inhalation and intravenous administration of the racemic drug results in high l-threo methylphenidate (l-MPH) serum levels. See, e.g., U.S. Pat. No. 6,395,752 to Midha et al., U.S. Pat. No. 6,127,385 to Midha et al.; U.S. Pat. No. 6,355,656 to Zeitlin, et al.; and U.S. Pat. No. 6,355,656 to Zeitlin, et al.

The present inventor has found that methylphenidate has a high abuse liability when administered non-medically, i.e., when the oral dosage formulations are crushed and snorted or dissolved and injected intravenously.

Methylphenidate is classified by the United States Food and Drug Administration as a Schedule II Controlled Substance. A Schedule II Controlled Substance is a drug that: (1) has a high potential for abuse; (2) has a currently accepted medical use in the United States, or a currently accepted medical use with severe restrictions; and (3) when abused, may lead to severe psychological or physical dependence. In addition to methylphenidate, Schedule II substances include morphine, PCP, cocaine, and methadone.

Methylphenidate is abused for the following stimulant effects: appetite suppression, increased focus and attention, wakefulness, and euphoria. To achieve the stimulant effect of methylphenidate, users choose modes of administration that bypass the presystemic metabolism (elimination) of the drug and facilitate rapid absorption of the drug into the users systemic circulation including the brain. As mentioned above, abusive modes of administration include (1) crushing one or more of the microdose tablets and snorting the powder and (2) dissolving the crushed tablets in a solution for intravenous injection. Adverse side effects from the abuse of methylphenidate are the same as those for other stimulants and include: loss of appetite; tremors and muscle twitching; fevers, convulsions, and headaches; anxiety and restlessness; paranoia, hallucinations, and delusions; excessive repetition of movements and meaningless tasks; formication (the sensation of bugs or worms crawling under the skin); and irregular heartbeat and respirations.

Studies on human subjects have found that different brain chemistries may indicate a patient's susceptibility to abuse a particular drug. For example, in a 1999 study, positron emission tomography was used to investigate the role of dopamine (DA) in the reinforcing effects of cocaine and methylphenidate in human subjects and its involvement in the susceptibility of a subject to become addicted to cocaine. The study demonstrated that the “high” experienced from the ingestion of these drugs is not caused by the mere presence of these drugs in the brain, rather, it is the result of the rate at which cocaine and methylphenidate enter the brain and block the dopamine transporters (DAT). The study showed that while a DAT blockade of greater than 50% is required for the drugs to induce a “high,” the rate of the DAT blockade determines whether the “high” is perceived or not. Thus, a slow DAT blockade will not induce a high even at doses that produce a DAT block in excess of 60%. Volkow N. D. et al. (December 1999), “Imaging Studies on the Role of Dopamine in Cocaine Reinforcement and Addiction in Human,” J. Psychopharmacol., 13(4):337-345; see also, Volkow N. D. et al. (January 1999), “Blockade of Striatal Dopamine Transporters by Intravenous Methylphenidate Is Not Sufficient to Induce Self-Reports of “High,” J. Pharmacol. Exp. Ther., 288(1): 14-20.

The Volkow et al. study indicates that oral MPH does not result in substance abuse because the rate of DAT blockade of the drug in this form is so slow that no “high” is perceived. By contrast, intravenous injection of MPH may result in abuse if the rate of DAT blockade in an individual subject is fast enough to produce a “high.”

Accordingly, there is a need for a method of reducing the likelihood that oral dosage forms of methylphenidate will be abused either by snorting or through intravenous administration. Such method can be achieved through the methylphenidate product of the present invention, alone or together with the pulsatile drug delivery system of the present invention.

Pharmaceutical dosage forms are known which provide a variety of drug release profiles, including immediate release, sustained release, and delayed release. That is, it may be desirable, for a particular drug, to prevent drug release after drug administration until a certain amount of time has passed (so-called “timed release”), to provide substantially continuous release over a predetermined time period (so-called “sustained release”) or to provide release immediately following drug administration (i.e., “immediate release”). For some types of drugs, it is preferred to release the drug in “pulses,” wherein a single dosage form provides for an initial dose of drug followed by a release-free interval, after which a second dose of drug is released, followed by one or more additional release-free intervals and drug release “pulses.” Pulsatile drug delivery is useful, for example, with active agents that have short half-lives and must be administered two or three times daily, with active agents that are extensively metabolized presystemically, and with active agents which lose the desired therapeutic effect over time when constant blood levels are maintained. These types of agents have pharmacokinetic-pharmacodynamic relationships that are best described by a clockwise “hysteresis loop.” A drug dosage form that provides a pulsatile drug release profile is also useful for minimizing the abuse potential of certain types of drugs, i.e., drugs for which tolerance, addiction and deliberate overdose can be problematic.

Because a precise and effective pulsatile drug delivery system is difficult to formulate and manufacture, there are few such dosage forms that have been commercialized. There are, however, several patents and literature references pertaining to pulsatile drug delivery. See, for example, U.S. Pat. No. 5,413,777 to Sheth et al., directed to a pulsatile once-a-day delivery system for the administration of minocycline; U.S. Pat. No. 5,260,068 to Chen, directed to a multiparticulate pulsatile drug delivery system; U.S. Pat. No. 4,777,049 to Magruder et al., directed to an osmotic delivery system for constant release of a drug with intermittent release “pulses”; U.S. Pat. No. 5,391,381 to Wong et al., directed to a drug dispenser for delivering individual drug-containing units in a “pulsatile” manner; PCT Publication No. WO 98/32424, pertaining to pulsatile delivery of diltiazem hydrochloride; U.S. Pat. Nos. 5,472,708 and 5,260,069 to Chen; Ishino et al. (1992) “Design and Preparation of Pulsatile Release Tablet as a New Oral Drug Delivery System,” Chem. Pharm. Bull. 40(11):3036-3041; Cohen et al. (1994), “Pulsatile Release from Microencapsulated Liposomes,” J. Liposome Res. 349-360; and Gazzaniga et al. (1994), “Chronotopic Drug Delivery Systems for Pulsatile and/or Site-Specific Release,” 21^{st}. Proc. Int. Symp. Controlled Release Bioact. Mater., pp. 744-745.

The present invention is directed in part to a novel method of preventing abuse of MPH in a methylphenidate responsive patient by incorporating an additional agent into the therapeutic oral dosage forms of MPH that is inert when the drug is taken orally but that produces unpleasant side effects when the drug is administered non-medically. The novel methylphenidate formulation may be administered through the novel pulsatile drug delivery system of the present invention, which is straightforward to manufacture and provides precisely timed drug release “pulses” at desired intervals. The pulsatile drug delivery system of the present invention provides a method for the oral administration of methylphenidate that ensures maximum therapeutic benefits of the drug with minimal adverse side effects and minimal abuse potential. To the best of applicants' knowledge, the method of preventing abuse of MPH and the pulsatile drug delivery system of the present invention are previously unknown and completely unsuggested by the art.

### SUMMARY OF THE INVENTION

Accordingly, it is a primary object of the invention to address the above-mentioned need in the art by providing a method for reducing the likelihood that a methylphenidate product will be abused by incorporating an emesis-inducing agent or a topical analgesic into the methylphenidate product, the emesis-inducing agent and the topical analgesic being inert when taken orally and inducing emesis or irritation only when the methylphenidate product is snorted or injected intravenously.

It is another object of the invention to treat a methylphenidate-responsive condition in a patient with methylphenidate while minimizing the likelihood that the patient will abuse or become addicted to the methylphenidate by first evaluating whether the patient has an elevated risk of abusing or becoming addicted to the methylphenidate, and if the determination is that the patient does have an elevated risk of abusing or becoming addicted to the methylphenidate, then treating the methylphenidate-responsive condition by orally administering to the patient a methylphenidate product that includes an emesis-inducing agent or a topical analgesic that is inert when taken orally and that only induces emesis or irritation when the methylphenidate product is snorted or injected intravenously.

It is another object of the invention to treating a methylphenidate-responsive condition in a patient with methylphenidate while minimizing the likelihood that the patient will abuse or become addicted to the methylphenidate by first evaluating whether the patient has an elevated risk of abusing or becoming addicted to the methylphenidate, and if the determination is that the patient does have an elevated risk of abusing or becoming addicted to the methylphenidate, then treating the methylphenidate-responsive condition by orally administering to the patient, once daily, a pulsatile release dosage form comprised of an immediate release dosage unit and a delayed release dosage unit, each said dosage unit containing a methylphenidate product, wherein following oral administration of the dosage form, a first dose of MPH is released substantially immediately from the immediate release dosage unit, followed by a time interval during which substantially no drug is released from the dosage form, and after which time interval a second dose of MPH is released from the delayed release dosage unit, and wherein the methylphenidate product includes an emesis-inducing agent or a topical analgesic that is inert when taken orally and that only induces emesis or irritation when the methylphenidate product is snorted or injected intravenously.

It is another object of the invention to treat a methylphenidate-responsive condition in a patient with methylphenidate while minimizing the likelihood that the patient will abuse or become addicted to the methylphenidate by first evaluating whether the patient has an elevated risk of abusing or becoming addicted to the methylphenidate, and if the determination is that the patient does have an elevated risk of abusing or becoming addicted to the methylphenidate, then treating the methylphenidate-responsive condition by orally administering to the patient, once daily, a pulsatile release dosage form comprised of an immediate release dosage unit and a delayed release dosage unit, each said dosage unit containing a methylphenidate product comprised of d-threo methylphenidate (d-MPH), wherein following oral administration of the dosage form, a first dose of d-MPH is released substantially immediately from the immediate release dosage unit, followed by a time interval during which substantially no drug is released from the dosage form, and after which time interval a second dose of d-MPH is released from the delayed release dosage unit, and wherein the methylphenidate product includes an emesis-inducing agent or a topical analgesic that are inert when taken orally and that only induce emesis or irritation when the methylphenidate product is snorted or injected intravenously.

It is another object of the invention to provide a methylphenidate product that comprises a therapeutic dose of racemic MPH or d-MPH together with an emesis-inducing agent such as apomorphine or the like or a topical analgesic such as capsaicin or the like.

It is a further object of the invention to provide a dosage form of oral methylphenidate wherein the drug has a ratio of d-MPH:l-MPH of at least 85:15, preferably, 90:10, more preferably 95:5, and most preferably 99:1.

It is yet another object of the invention to provide a dosage form wherein the MPH is released in three timed intervals.

It is still a further object of the invention to provide such a dosage form comprising at least two individual drug-containing dosage units, each of which has a different drug release profile.

It is another object of the invention to provide a dosage form wherein the dosage units are housed in a closed capsule.

It is a further object of the invention to provide a dosage form wherein the dosage units are compressed tablets.

It is yet another object of the invention to provide such a dosage form wherein the dosage units are drug-containing particles or beads.

It is still a further object of the invention to provide such a dosage form comprised of a single tablet of which the drug-containing dosage units represent integral but discreet segments.

It is another object of the invention to provide such a dosage form for administering methylphenidate optionally in combination with one or more other active agents such as CNS stimulants (including analeptic agents and psychostimulants), antidepressant drugs, antianxiety drugs, and the like.

It is a further additional object of the invention to provide methods for administering methylphenidate optionally in combination with one or more other active agents such as analgesics, including but not limited to, aspirin, acetaminophen, ephedrine, psuedoephedrine and the like.

It is an additional object of the invention to provide methods for administering methylphenidate using the novel dosage forms.

Additional objects, advantages, and novel features of the invention will be set forth in part in the description of the invention which follows, and in part will become apparent to those skilled in the art upon examination of the following, or may be learned by practice of the invention.

### BRIEF DESCRIPTION OF THE DRAWINGS

FIG. 1 shows a graph of mean plasma concentration versus time plots of d-threo methylphenidate (triangles) and l-threo methylphenidate (circles) in 11 healthy male subjects after intravenous (IV) (closed) or immediate release (IR) oral (open) administration of methylphenidate.

FIG. 2A shows a graph of dose by time for two different continuous dosing schedules (CD1 and CD2) of racemic methylphenidate in two groups (n=9 and n=11, respectively) of 10-11 year old boys with methylphenidate-responsive ADHD.

FIG. 2B shows a graph of dose by time for two different pulsatile dosing schedules (PD1 and PD2) of racemic methylphenidate in two groups (both groups having n=10) of 10-11 year old boys with methylphenidate-responsive ADHD.

FIG. 3A shows a graph of the pharmacokinetic profile of d-threo methylphenidate levels following administration of racemic methylphenidate in dosing schedules CD1 and CD2.

FIG. 3B shows a graph of the pharmacokinetic profile of d-threo methylphenidate levels following administration of racemic methylphenidate in dosing schedules PD1 and PD2.

FIG. 4A shows a graph of the percent improvement over a twelve hour period of time in the attention of the two groups of boys referenced in FIG. 2A after administration of methylphenidate in dosing schedules CD1 and CD2.

FIG. 4B shows a graph of the percent improvement over a twelve hour period of time in the attention of the two groups of boys referenced in FIG. 2B after administration of methylphenidate in dosing schedules PD1 and PD2.

FIG. 5A shows a graph of the percent improvement over a twelve hour period of time in the activity of the two groups of boys referenced in FIG. 2A after administration of methylphenidate in dosing schedules CD1 and CD2.

FIG. 5B shows a graph of the percent improvement over a twelve hour period of time in the activity of the two groups of boys referenced in FIG. 2B after administration of methylphenidate in dosing schedules PD1 and PD2.

### DETAILED DESCRIPTION OF THE INVENTION

### I. Overview and Definitions

It is to be understood that unless otherwise indicated, this invention is not limited to specific active agents, vehicles, excipients, dosage forms, or the like, as such may vary. It is also to be understood that the terminology used herein is for describing particular embodiments only, and is not intended to be limiting.

As used in this specification and the appended claims, the singular forms “a,” “an,” and “the” include plural referents unless the context clearly dictates otherwise. Thus, for example, reference to “an active agent” includes a single active agent as well a two or more different active agents in combination, reference to “a pharmaceutically acceptable carrier” includes mixtures of two or more such carriers as well as a single carrier, and the like.

In describing and claiming the present invention, the following terminology will be used in accordance with the definitions set out below.

The terms “active agent,” “pharmacologically active agent,” and “drug” are used interchangeably herein to refer to a chemical compound, complex or composition that is intended to have a beneficial biological effect but may be associated with adverse effects as well, the beneficial biological effect is preferably a therapeutic effect in the treatment of a disease or abnormal physiological condition. The terms also encompass pharmaceutically acceptable, pharmacologically active derivatives of those active agents specifically mentioned herein, including, but not limited to, salts, esters, amides, prodrugs, active metabolites, isomers, fragments, analogs, and the like. When the terms “active agent,” “pharmacologically active agent” and “drug” are used, then, or when a particular active agent is specifically identified, it is to be understood that the term includes the active agent per se as well as pharmaceutically acceptable, pharmacologically active salts, esters, amides, prodrugs, active metabolites, isomers, fragments, analogs, etc.

When the term “methylphenidate” (also referred to as MPH) is used herein without further qualification, the term refers to a racemic mixture of d-threo-methylphenidate (d-MPH) and l-threo methylphenidate (l-MPH). If a single enantiomer of methylphenidate is intended, the agent will be referred to as either d-threo methylphenidate or l-threo methylphenidate, or their defined abbreviations.

“Substantially free of l-MPH” refers to a pharmaceutical composition of methylphenidate that has a ratio of d-MPH:l-MPH of at least 85:15, preferably 90:10, more preferably 95:5, and most preferably 99:1.

The term “dosage form” denotes any form of a pharmaceutical composition that contains an amount of active agent sufficient to achieve a therapeutic effect.

The terms “treating” and “treatment” as used herein with respect to treatment of a patient refer to reduction in severity and/or frequency of symptoms, elimination of symptoms and/or underlying cause, and prevention of the occurrence of symptoms and/or their underlying cause, and improvement or remediation of damage. For example, treatment of a methylphenidate-responsive condition in a patient using the method of the invention encompasses prevention of the methylphenidate-responsive condition in a patient susceptible to developing the condition (e.g., at an elevated risk of developing the condition, as a result of genetic predisposition, environmental factors, or the like) as well as treatment of a patient who exhibits symptoms of the condition.

By an “effective amount,” a “pharmaceutically effective amount,” and a “therapeutically effective amount” of an active agent is meant a nontoxic but sufficient amount of the agent to provide the desired effect. The exact amount of active agent that is “effective” will vary from subject to subject, depending on the age and general condition of the individual, the severity of the condition being treated, and the like. Thus, it is not always possible to specify an exact “effective amount.” However, an appropriate “effective” amount in any individual case may be determined by one of ordinary skill in the art using routine experimentation

By “pharmaceutically acceptable,” as in the recitation of a “pharmaceutically acceptable carrier,” or a “pharmaceutically acceptable additive,” is meant a material that is not biologically or otherwise undesirable, i.e., the material may be incorporated into a pharmaceutical composition administered to a patient without causing any undesirable biological effects or interacting in a deleterious manner with any of the other components of the composition in which it is contained. “Pharmacologically active” (or simply “active”), as in a “pharmacologically active” derivative of an active agent, refers to a derivative having the same type of pharmacological activity as the parent compound and approximately equivalent in degree. When the term “pharmaceutically acceptable” is used to refer to a derivative (e.g., a salt) of an active agent, it is to be understood that the compound is pharmacologically active as well. When the term “pharmaceutically acceptable” is used to refer to an excipient, it implies that the excipient has met the required standards of toxicological and manufacturing testing or that it is on the Inactive Ingredient Guide prepared by the FDA.

The term “delayed release” is used to refer to a drug dosage unit in which there is a time delay provided between oral administration of a dosage form containing the dosage unit and the release of the drug.

“Carriers” refer to conventional pharmaceutically acceptable vehicles suitable for incorporation into an oral dosage form, and include any such materials known in the art that are nontoxic and do not interact with other components of a dosage form or dosage unit in a deleterious manner.

“Non-medical use” of methylphenidate refers to the non-prescription use of methylphenidate that includes, but is not limited to, the ingestion of methylphenidate by crushing methylphenidate tablets for snorting or by dissolving the tablets in a solution for injection.

“Illicit drug use” refers to any illegal use of medications and includes, but is not limited to, non-medical use of prescription medications and the buying and selling of prescription medications for non-medical use.

“Addiction” to methylphenidate refers to the state in which a subject engages repeatedly in non-medical use and/or illicit use of methylphenidate.

“Elevated risk for abuse” refers to the likelihood that a person having a methylphenidate-responsive condition will abuse, become addicated to the methylphenidate, or encourage others to abuse the drug by selling the prescribed methylphenidate to others for non-medical use.

“Evaluating the risk for abuse” refers to the procedures for determining the likelihood that the person with the methylphenidate-responsive condition will abuse or become addicted to methylphenidate. Such procedures include psychological evaluations. While determining the rate of the DAT blockade of subjects to intravenously administered methylphenidate may be cost-prohibitive in most situations, such a determination may be another tool to determine abuse liability in some subjects.

### II. Evaluating the Risk of Methylphenidate Abuse

As previously discussed, it is known that a high rate of DAT blockade causes the “high” associated with the abuse of MPH. Abuse of methylphenidate includes such non-medical uses as crushing methylphenidate tablets and inhaling or snorting the resulting powder or dissolving the crushed tablets in a solution for parenteral or intravenous injection. Abuse of methylphenidate can lead to addiction and other illicit uses of methylphenidate, such as inducing others to use the drug by selling the methylphenidate for money.

In one embodiment of the present invention, a method is provided for treating a methylphenidate-responsive condition in a patient with methylphenidate while minimizing the likelihood that the patient will abuse or become addicted to the methylphenidate, by first determining whether the patient has an elevated risk of abusing or becoming addicted to methylphenidate. The patient's susceptibility to abuse or become addicted to methylphenidate is determined through psychological evaluations. If the patient is over the age of 18, the evaluations should include, but need not be limited to: a psychological profile to identify factors contributing to an elevated risk of drug abuse or addiction; an evaluation of the patient's history of drug abuse or addiction; and an evaluation of the patient's family history of drug abuse or addiction. If the patient is between the ages of 9 to 17 years of age, all of the foregoing evaluations may be applied; however, the determination of susceptibility to abuse or addiction should not based solely on the psychological profile as the young age of the patient may not provide a complete profile by itself. The psychological profile, however, may assist in determining if the methylphenidate-responsive 9-17 year old has a substantially greater likelihood of abusing or becoming addicted to methylphenidate relative to the likelihood that an average patient in a population pool of patients of ages 9-17 years of age will abuse or become addicted to methylphenidate. If the patient is 9 years old or younger, the psychological profiles and history of abuse or addiction evaluations may be insufficient to provide a determination of susceptibility for abuse or addiction. Accordingly, with patients aged under the age of 9, determinations should be based upon an evaluation of the patient's family history of abuse.

Example 1 sets forth one procedure that may be used to determine the abuse liability of a person over the age of 18. This procedure is not intended for any patients under the age of 18. Under the protocol set forth in Example 1, it can be determined whether a methylphenidate-responsive patient has a substantially greater likelihood of abusing or becoming addicted to methylphenidate relative to the likelihood that an average patient in a population pool of patients at least 18 years of age will abuse or become addicted to methylphenidate.

### III. Methylphenidate Formulations

Once the methylphenidate-responsive patient is determined to have an elevated risk of abusing or being addicted to methylphenidate, the patient is treated with an oral dosage form of methylphenidate that has an additional agent that is inert when taken orally but that causes unpleasant side effects when the oral dosage forms are abused either by crushing the drug for snorting or dissolving the drug for intravenous injection. Suitable additional agents include, but are not limited to, emesis-inducing agents such as apomorphine or the like, which are inert when taken orally and emesis-inducing when taken through non-medical administration, or topical analgesics such as capsaicin or the like, which are slightly irritating when taken orally and irritants when taken non-medically. The unpleasant side effects caused by the addition of the emesis-inducing agent or the topical analgesic in the oral form of the drug will inhibit the abuse of the drug by making the non-medical administration of the drug extremely unpleasant.

The amount of apomorphine that can be safely incorporated into the oral dosage form of MPH is 1.5 to 7 mg, with 5 mg being an acceptable amount to safely induce emesis without causing the subject severe distress. For safety reasons, no more than 10 mg of an emesis-inducing agent should be included in the total MPH oral dosage form. The table in Example 3 shows a typical formulation for “abuse-free” methylphenidate prepared with 5 mg of apomorphine.

Other suitable topical analgesics in the same family as capsaicin that may also be used in preparing the “abuse free” dosage form of MPH include, but are not limited to, capsaicin oleoresin (an alcoholic resin of capsaicin) and nonivamide (also referred to as “synthetic capsaicin”). When topical analgesics are included in the total MPH oral dosage form in an amount approximately equivalent to 0.5% wt., the subject will experience irritation without severe distress. The table in Example 3 shows a typical formulation for “abuse-free” methylphenidate prepared with 0.5% wt. of capsaicin.

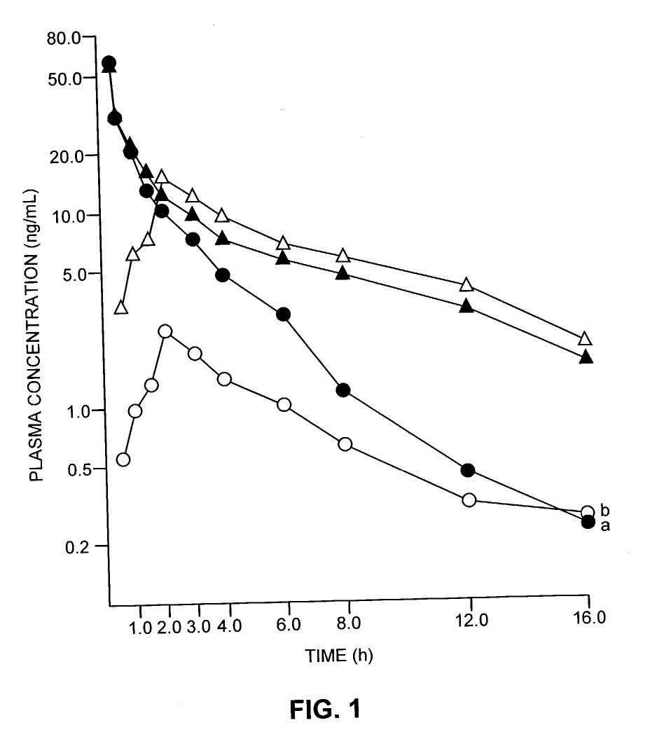

The present inventors have found that after oral administration of commercially available racemic methylphenidate, the bioavailability of d-MPH in blood plasma is approximately 22% (so that approximately 78% of the d-MPH is metabolized) and the bioavailability of l-MPH in plasma is 5% (so that approximately 95% is metabolized). The inventors have also found that when methylphenidate is administered intravenously, there are approximately equivalent plasma levels of both d-MPH and l-MPH up to 1.5 hours post dose. FIG. 1 shows a concentration versus time plot of d-MPH and l-MPH after oral and intravenous administration of racemic methylphenidate. It is believed that as the levels of l-MPH in the blood decreases, the adverse side effects associated with the therapeutic dosage forms of orally administered MPH decreases.

Thus, to alleviate the risk of adverse side effects associated with the presence of the l-threo enantiomer, the drug may be administered in a dosage form that is substantially free of l-MPH. MPH that is substantially free of l-MPH has a ratio of d-MPH:l-MPH of at least 85:15, with formulations having ratios of d-MPH to l-MPH of at least 90:10, 95:5, and 99:1 being preferred.

To maximize the effect of the oral MPH, the drug may be administered in a once-daily pulsatile release dosage form comprised of an immediate release dosage unit and a delayed release dosage unit, each said dosage unit containing MPH and at least one of said dosage units containing the additional emesis-inducing agent. This mode of administration of MPH has the advantage of increasing the efficacy of the orally administered MPH by ensuring that effective dosages of d-MPH are released into the patient's system throughout a set time interval. Under the pulsatile release delivery system, a first dose of MPH is released substantially immediately from the immediate release dosage unit, followed by a time interval during which substantially no drug is released from the dosage form, and after which time interval a second dose of MPH is released from the delayed release dosage unit.

By “pulsatile” is meant that a plurality of drug doses are released at spaced apart time intervals. Generally, upon ingestion of the dosage form, release of the initial dose is substantially immediate, i.e., the first drug release “pulse” occurs within 1-2 hours of ingestion. This initial pulse is followed by a first time interval during which substantially no drug is released from the dosage form, after which a second dose is then released. Typically, the second dose is released on the order of 3-5 hours following ingestion of the dosage form. Preferably, release of the second dose is followed by a second non-release interval, which is again followed by a “pulse” of drug release. Ideally, release of a third dose occurs on the order of 7-9 hours following ingestion. In a preferred embodiment herein, either two or three release pulses are provided. However, the invention is also intended to encompass dosage forms that provide more than three pulses, with non-release intervals therebetween of approximately 2-6 hours, preferably 3-5 hours. Example 6 shows the superiority of the pulsatile dosing system of the present invention over more traditional continuous dosing systems.

The aforementioned pulsatile release profile is achieved with dosage forms that, in one embodiment, are closed and preferably sealed capsules housing two or more drug-containing “dosage units.” In a preferred embodiment, each dosage unit comprises a compressed or molded tablet, wherein each of the tablets within the capsule provides a different drug release profile. That is, for an exemplary dosage form, a first tablet releases drug substantially immediately following ingestion of the dosage form, while a second tablet in the capsule releases drug approximately 3-5 hours following ingestion, and an optional third tablet provides drug release after approximately 7-9 hours. While the dosage form will not generally include more than three tablets, dosage forms housing four or more tablets are within the scope of the present invention.

In an alternative embodiment, each dosage unit comprises a drug-containing particle or bead (drug-containing “beads” refer to drug-coated inert supports, e.g., lactose beads coated with drug). A first group of these particles or beads releases drug substantially immediately following ingestion of the dosage form, a second group releases drug approximately 3-5 hours following ingestion, and an optional third group provides drug release after approximately 7-9 hours.

In a further alternative embodiment, the individual dosage units are compacted in a single tablet, and represent integral but discrete segments thereof (e.g., layers). For example, drug-containing particles or drug-containing beads can be compressed together into a single tablet using conventional tabletting means.

As will be appreciated by those skilled in the art and as described in the pertinent texts and literature, a number of methods are available for preparing drug-containing tablets or other dosage units, which provide a variety of drug release profiles. Such methods include coating a drug or drug-containing composition, increasing the drug's particle size, placing the drug within a matrix, and forming complexes of the drug with a suitable complexing agent.

The delayed release dosage units in the present capsules can be prepared, for example, by coating a drug or a drug-containing composition with a selected membrane coating material, typically although not necessarily a polymeric material. When a coating is used to provide delayed release dosage units, particularly preferred coating materials comprise bioerodible, gradually hydrolyzable and/or gradually water-soluble polymers. The “coating weight,” or relative amount of coating material per dosage unit, generally dictates the time interval between ingestion and drug release.

Suitable membrane coating materials for effecting delayed release include, but are not limited to: cellulosic polymers such as hydroxypropyl cellulose, hydroxyethyl cellulose, hydroxypropyl methyl cellulose, methyl cellulose, ethyl cellulose, cellulose acetate, cellulose acetate phthalate, cellulose acetate trimellitate, hydroxypropylmethyl cellulose phthalate, cellulose ester-ether phthalate, hydroxypropylcellulose phthalate, alkali salts of cellulose acetate phthalate, alkaline earth salts of cellulose acetate phthalate, hydroxypropylmethyl cellulose hexahydrophthalate, cellulose acetate hexahydrophthalate, and carboxymethylcellulose sodium; acrylic acid polymers and copolymers preferably formed from acrylic acid, methacrylic acid, acrylic acid alkyl esters, methacrylic acid alkyl esters, and the like, e.g. copolymers of acrylic acid, methacrylic acid, methyl acrylate, ethyl acrylate, methyl methacrylate and/or ethyl methacrylate, with a terpolymer of ethyl acrylate, methyl methacrylate and trimethylammonioethyl methacrylate chloride (sold under the tradename Eudragit RS) particularly preferred; vinyl polymers and copolymers such as polyvinyl pyrrolidone, polyvinyl acetate, polyvinylacetate phthalate, vinylacetate crotonic acid copolymer, and ethylene-vinyl acetate copolymers; and shellac, ammoniated shellac, shellac-acetyl alcohol, and shellac n-butyl stearate.

In some cases, it may be desirable for the third tablet or bead or particle fraction to provide for release of the active agent in the colon, in which case polymeric or other materials are used that enable drug release within the colon. These may be selected from the aforementioned list, or other materials may be used as will be known to those skilled in the art of pharmaceutical formulation and drug delivery. For example, hydrocolloid gums may be effective to provide for colonic delivery, e.g., guar gum, locust gum, bena gum, gum tragacanth, and karaya gum (see, e.g., U.S. Pat. No. 5,656,294 to Friend). Other materials suitable for effecting colonic drug delivery include polysaccharides, mucopolysaccharides, and related compounds, e.g., pectin, arabinogalactose, chitosan, chondroitin sulfate, dextran, galactomannan, and xylan. Combinations of different coating materials may also be used to coat a single dosage unit.

To bring about the desired pulsatile release profile for a dosage form comprised of encapsulated tablets, the first tablet is provided with little or no coating material, the second tablet is provided with some degree of coating material, the coating weight of a third tablet is still higher, and so on. Analogously, for encapsulated dosage forms in which the drug-containing dosage units are beads or particles, a first fraction of beads or particles is provided with little or no coating material, a second fraction is provided with some degree of coating material, the coating weight of a third fraction is still higher, etc. For example, when the dosage form contains three tablets (or, analogously, three groups of drug-containing particles or beads), the first tablet, which releases drug substantially immediately, may have a total coating weight of less than about 10%, preferably less than about 8%, the second tablet may have a total coating weight in the range of approximately 10% to 30%, preferably 15% to 25%, and the third tablet, if present, may have a total coating weight in the range of approximately 15% to 65%, preferably 20% to 65%. The preferred coating weights for particular coating materials may be readily determined by those skilled in the art by evaluating individual release profiles for dosage units prepared with different quantities of various coating materials.

Alternatively, the delayed release dosage units, i.e., tablets or particles, may be formulated by dispersing the drug within a matrix of a suitable material such as an insoluble plastic, a hydrophilic polymer, or a fatty compound. The insoluble plastic matrices may be comprised of, for example, polyvinyl chloride or polyethylene. Hydrophilic polymers useful for providing a matrix for a delayed release dosage unit include, but are not limited to, those described above as suitable coating materials. Fatty compounds for use as a matrix material include, but are not limited to, waxes generally (e.g., carnauba wax) and glyceryl tristearate. Once the active ingredient is mixed with the matrix material, the mixture can be compressed into tablets or processed into individual drug-containing particles.

The individual dosage units may be provided with colored coatings, with a single color used to identify a tablet or bead or particle fraction having a corresponding delayed release profile. That is, for example, a blue coating may be used for the immediate release tablet or bead or particle fraction, a red coating may be used for the “medium” release tablet or bead or particle fraction, and the like. In this way, errors during manufacture can be easily avoided. The color is introduced by incorporating a pharmaceutically acceptable colorant into the coating during coating preparation. The colorant may be either natural or synthetic. Natural colorants include pigments such as chlorophyll, anattenes, beta-carotene, alizarin, indigo, rutin, hesperidin, quercitin, carminic acid, and 6,6-dibromoindigo. Synthetic colorants are dyes, including both acidic dyes and basic dyes, such as nitroso dyes, nitro dyes, azo dyes, oxazines, thiazines, pyrazolones, xanthenes, indigoids, anthraquinones, acridines, rosanilines, phthaleins, quinolines. e.g., a dye or pigment, during preparation of the coating solution.

For encapsulated tablets, the weight of each individual tablet in the capsule is typically in the range of about 10 mg to 150 mg, preferably in the range of about 25 mg to about 100 mg, and most preferably is in the range of about 40 mg to 80 mg. The individual tablets are prepared using conventional means. A preferred method for forming tablets herein is by direct compression of a powdered, crystalline or granular drug-containing composition, alone or in combination with diluents, binders, lubricants, disintegrants, colorants or the like. As an alternative to direct compression, compressed tablets can be prepared using wet-granulation or dry-granulation processes. Tablets may also be molded rather than compressed, starting with a moist material containing a suitable water-soluble lubricant. Preferred tablets herein are manufactured using compression rather than molding, however. Drug-containing particles or beads are also prepared using conventional means, typically from a fluid dispersion.

Conventional coating procedures and equipment may then be used to coat the dosage units, i.e., the drug-containing tablets, beads or particles. For example, a delayed release coating composition may be applied using a coating pan, an airless spray technique, fluidized bed coating equipment, or the like. For detailed information concerning materials, equipment and processes for preparing tablets, beads, drug particles, and delayed release dosage forms, reference may be had to Pharmaceutical Dosage Forms: Tablets, eds. Lieberman et al. (New York: Marcel Dekker, Inc., 1989), and to Ansel et al., Pharmaceutical Dosage Forms and Drug Delivery Systems, 6th Ed. (Media, Pa.: Williams & Wilkins, 1995).

Optional components present in the individual drug-containing dosage units include, but are not limited to, diluents, binders, lubricants, disintegrants, stabilizers, surfactants, coloring agents, and the like. Diluents, also termed “fillers,” are typically necessary to increase the bulk of a tablet so that a practical size is provided for compression. Suitable diluents include, for example, dicalcium phosphate dihydrate, calcium sulfate, lactose, cellulose, kaolin, mannitol, sodium chloride, dry starch, hydrolyzed starches, silicon dioxide, titanium oxide, alumina, talc, microcrystalline cellulose, and powdered sugar. Binders are used to impart cohesive qualities to a tablet formulation, and thus ensure that a tablet remains intact after compression. Suitable binder materials include, but are not limited to, starch (including corn starch and pregelatinized starch), gelatin, sugars (including sucrose, glucose, dextrose, lactose and sorbitol), polyethylene glycol, waxes, natural and synthetic gums, e.g., acacia, tragacanth, sodium alginate, polyvinylpyrrolidone, celluloses, and Veegum, and synthetic polymers such as polymethacrylates and polyvinylpyrrolidone. Lubricants are used to facilitate tablet manufacture; examples of suitable lubricants include, for example, magnesium stearate, calcium stearate, stearic acid, glyceryl behenate, and polyethylene glycol, and are preferably present at no more than approximately 1 wt. % relative to tablet weight. Disintegrants are used to facilitate tablet disintegration or “breakup” after administration, and are generally starches, clays, celluloses, algins, gums or crosslinked polymers. Stabilizers are used to inhibit or retard drug decomposition reactions, which include, by way of example, oxidative reactions. Surfactants may be anionic, cationic, amphoteric, or nonionic surface-active agents, with anionic surfactants preferred. Suitable anionic surfactants include, but are not limited to, those containing carboxylate, sulfonate and sulfate ions, associated with cations such as sodium, potassium and ammonium ions. Particularly preferred surfactants include, but are not limited to: long alkyl chain sulfonates and alkyl aryl sulfonates such as sodium dodecylbenzene sulfonate; dialkyl sodium sulfosuccinates, such as sodium bis-(2-ethylhexyl)-sulfosuccinate; and alkyl sulfates such as sodium lauryl sulfate. If desired, the tablets may also contain minor amounts of nontoxic auxiliary substances such as wetting or emulsifying agents, pH buffering agents, preservatives, and the like.

As noted earlier herein, in one embodiment, the individual drug tablets, beads, or particles are contained within a closed capsule. The capsule material may be either hard or soft, and as will be appreciated by those skilled in the art of pharmaceutical science, typically comprises a tasteless, easily administered and water soluble compound such as gelatin, starch or cellulose. A preferred capsule material is gelatin. The capsules are preferably sealed, such as with gelatin bands or the like. See, for example, Remington: The Science and Practice of Pharmacy, Nineteenth Edition (Easton, Pa.: Mack Publishing Co., 1995), which describes materials and methods for preparing encapsulated pharmaceuticals designed to dissolve shortly after ingestion.

For both the methylphenidate that is pure d-MPH and the methylphenidate that is substantially free of l-MPH, the total d-MPH in the dosage form is in the range of approximately 1 mg to 100 mg, with 2 mg to 50 mg preferred, and 0.5 mg to 20 mg more preferred. Optimally, the first and second dosage units each contain approximately 0.5 to 20 mg d-MPH, and preferably, the first and second methylphenidate doses are approximately the same. The third tablet should contain a lower dose of methylphenidate, preferably about half the dose in the first tablet, to avoid sleep disruption. If an additional CNS stimulant such as d-amphetamine is incorporated into the dosage form, it will be included in the first, immediate release dosage unit; will optionally be present in the second dosage unit (and if present, at a lower dose than in the first dosage unit); and will not be included in the third dosage unit. Example 4 shows the preparation of pure d-MPH tablets and Example 5 shows the preparation of “adverse effect free” methylphenidate tablets having a d-MPH:l-MPH ratio of 99:1.

It may be desirable to include one or more additional active agents in the dosage forms herein. These active agents may potentiate certain effects of methylphenidate, or vice versa. The additional active agent or agents may be combined with methylphenidate in a single dosage unit within the dosage form, or one or more dosage units within the dosage form may comprise the additional active agent without any methylphenidate. In the former case, the various active agents may be present as an admixture in a tablet, or the agents may be physically segregated as in a bilayer tablet, a tablet having two or more active agent-containing coatings, or the like.

As mentioned previously, additional agents to the oral dosage form of MPH can include abuse-inhibiting agents. One class of abuse-inhibiting agents includes emesis-inducing agents such as apomorphine, which is inert in oral form and emesis inducing only when snorted or taken intravenously. Another class of abuse-inhibiting agents includes topical analgesics such as capsaicin, which is also inert in its oral form and a strong irritant when snorted or taken intravenously. It is understood that the abuse-free embodiment of the present invention is not limited to the use of emesis-inducing agents or topical analgesics, but may include any agents that are inert in oral form and that produce unpleasant side effects when abused via snorting or intravenous injection.

Other preferred additional active agents, i.e., active agents for co-administration with methylphenidate, are CNS stimulants (including analeptic agents and psychostimulants), antidepressant drugs, and antianxiety agents. Particularly preferred are CNS stimulants including, but not limited to: amphetamine (racemic), d-amphetamine, amphetamine and d-amphetamine phosphate, amphetamine and d-amphetamine sulfate, amphetamine and d-amphetamine hydrochloride, amphetamine and d-amphetamine saccharate, and amphetamine and d-amphetamine aspartate, amphetaminil, bemegride, benzphetamine, benzphetamine hydrochloride, brucine, chlorphentermine, clofenciclan, clortermine, deanol acetamidobenzoate, demanyl phosphate, dexoxadrol, diethylpropion, doxapram hydrochloride, N-ethylamphetamine, ethamivan, etifelmin, etryptamine, fencamfamine, fenethylline, fenosolone, fenfluramine, flurothyl, hexacyclonate sodium, homocamfin, mazindol, megexamide, methamphetamine, d-methamphetamine, nicotinic agonists, nikethamide, pemoline, pentylenetetrazole, phenidimetrazine, phendimetrazine tartrate, phenmetrazine, phenmetrazine hydrochloride, phentermine, picrotoxin, pipradrol, pipradrol hydrochloride, prolintane, pyrovalerone, racephedrine, racephedrine hydrochloride, and tetrahydrobenzothienopyridines. Pemoline, amphetamine, d-amphetamine and salts thereof are particularly preferred additional active agents.

Antidepressant drugs include, for example: tricyclic antidepressants such as imipramine, amitryptyline, amoxapine, clomipramine, desipramine, doxepin, imipramine, maprotiline, nortriptyline, protriptyline and trimipramine; monoamine oxidase inhibitors (MAOIs) such as isocarboxazid, phenelzine, selegiline and tranylcypromine; selective serotonin reuptake inhibitors (SSRIs) such as fluoxetine, fluvoxamine, paroxetine, sertraline, venlaxafine, citalopram, s-citalopram (escitalopram), and the like; and other antidepressants including buproprion, nefazodone and trazodone.

Examples of antianxiety agents include, but are not limited to, benzodiazepines such as alprazolam, chlordiazepoxide, clonazepam, clorazepate, diazepam, estazolam, flurazepam, halazepam, lorazepam, midazolam, oxazepam, prazepam, quazepam, temazepam and triazolam; carbamates such as meprobamate and ethinamate; chloral hydrate and related drugs; piperidine-diones such as glutethimide and methyprylon; alcohols such as ethchlorvynol; antihistaminics such as diphenhydramine and hydroxyzine; methaqualone; and paraldehyde.

Other additional active agents may include analgesics such as aspirin, acetaminophen, pseudoephedrine, and the like. These additional agents may be useful for administration to methylphenidate-responsive patients whose conditions are associated with pain or flu-like symptoms.

Each of the active agents in the individual tablets may be in the form of a pharmaceutically acceptable salt, ester, amide, prodrug or other derivative or analog, including active agents modified by appending one or more appropriate functionalities to enhance selected biological properties. Such modifications are known in the art and/or are described in the pertinent texts and literature.

Salts of the active agents used in conjunction with the present dosage forms may be obtained commercially or can be prepared using standard procedures known to those skilled in the art of synthetic organic chemistry and described, for example, by J. March, Advanced Organic Chemistry: Reactions, Mechanisms and Structure, 4th Ed. (New York: Wiley-Interscience, 1992). Suitable acids for preparing acid addition salts may be weak acids, medium acids, or strong acids, and include both organic acids, e.g., acetic acid, propionic acid, glycolic acid, pyruvic acid, oxalic acid, malic acid, malonic acid, succinic acid, maleic acid, fumaric acid, aspartic acid, saccharic acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. Preparation of basic salts of acid moieties which may be present (e.g., carboxylic acid groups) are prepared using a pharmaceutically acceptable base such as sodium hydroxide, potassium hydroxide, ammonium hydroxide, calcium hydroxide, magnesium hydroxide, trimethylamine, or the like. Preparation of esters involves functionalization of hydroxyl and/or carboxyl groups that may be present. These esters are typically acyl-substituted derivatives of free alcohol groups, i.e., moieties that are derived from carboxylic acids of the formula RCOOH where R is alkyl, and preferably is lower alkyl. Pharmaceutically acceptable esters may be prepared using methods known to those skilled in the art and/or described in the pertinent literature. Amides, prodrugs, and other analogs and derivatives can be readily prepared as well, using conventional means.

### IV. Utility

The novel method of the present invention improves upon the current administration of methylphenidate in two ways. First, it prevents the abuse of methylphenidate by making the non-medical use of the drug an unpleasant experience and second, it makes the therapeutic administration of methylphenidate more pleasant by reducing the adverse side effects found in the commercially available formulations. “Abuse-free” methylphenidate products are comprised by incorporating an emesis-inducing agent or a topical analgesic into the methylphenidate product, the emesis-inducing agent and the topical analgesic being inert in the oral form and emesis-inducing or irritating, respectively, when the MPH is taken non-medically. “Adverse-free” methylphenidate products are comprised by formulating the methylphenidate product so that it contains practically no l-MPH.

According to the present invention, abuse liability of the methylphenidate-responsive patient is determined by through psychological evaluations, which include, but are not limited to, evaluating the patient's psychological profile to identify factors contributing to an elevated risk of drug abuse or addiction; evaluating the patient's history of drug abuse or addiction; and/or evaluating the patient's family history of drug abuse or addiction. If the methylphenidate-responsive patient is found to be susceptible to abuse or addiction of methylphenidate, then that patient is to be administered the novel “abuse-free” methylphenidate product of the present invention.

The novel drug dosage forms are to be administered orally to a methylphenidate-responsive patient to treat or prevent a variety of disorders, conditions, and diseases. In accordance with the present invention, administration of methylphenidate may be carried out in order to treat any disorder, condition or disease for which methylphenidate is generally indicated. Such disorders, conditions and diseases include, for example, ADD, ADHD, narcolepsy, chronic fatigue syndrome, bulimia, and acute depression; methylphenidate may also be used in the treatment of individuals suffering from cognitive decline associated with AIDS or AIDS-related conditions, and for mood elevation in terminally ill patients suffering from a disease such as cancer.

It is to be understood that while the invention has been described in conjunction with the preferred specific embodiments thereof, that the description above as well as the examples that follow are intended to illustrate and not limit the scope of the invention. Other aspects, advantages, and modifications within the scope of the invention will be apparent to those skilled in the art to which the invention pertains.

### V. Experimental Analysis

The practice of the present invention will employ, unless otherwise indicated, conventional techniques of pharmaceutical formulation, medicinal chemistry, biological testing, and the like, which are within the skill of the art. Such techniques are explained fully in the literature. Preparation of various types of pharmaceutical formulations are described, for example, in Lieberman et al., cited supra; synthesis of chiral drugs is described, inter alia, in Wilson and Gisvold, Textbook of Organic, Medicinal and Pharmaceutical Chemistry (Lippincott-Raven Publishers, 1991); and Gibaldi and Perrier, Pharmacokinetics (Marcel Dekker, 1982), provides a description of the biological testing procedures useful to evaluate compounds such as those described and claimed herein. All patents, patent applications, and publications mentioned herein, both supra and infra, are hereby incorporated by reference.

In the following examples, efforts have been made to ensure accuracy with respect to numbers used (e.g., amounts, temperature, etc.) but some experimental error and deviation should be accounted for. Unless indicated otherwise, temperature is in degrees C. and pressure is at or near atmospheric. All reagents were obtained commercially unless otherwise indicated.

### EXAMPLE 1

### Abuse Liability Protocol

Objective:

The abuse liabilities of intravenous d-threo-methylphenidate (20 mg), intravenous l-threo-methylphenidate (20 mg) and oral d-amphetamine (30 mg) are compared with an intravenous placebo (saline injection) plus an oral placebo (lactose) in a double blind, 4-period, randomized cross-over design.

Inclusion Criteria:

The study is conducted in a cohort of 24 healthy men and women aged 18-45 years who, in the previous two years, have a history of at least four episodes of recreational stimulant drug abuse as determined by the Alcohol and Drug Abuse Research Center background questionnaire (ADARC-III). The subjects are otherwise required to be in good physical and mental health as confirmed by medical history, physical and psychiatric examinations. For women subjects, a pregnancy test using a medically acceptable form of contraception is required if the subject is sexually active. During the course of the study, all women must agree to avoid becoming pregnant. For all subjects, informed consent in the form of agreement to complete all study ratings, screening procedures, and post study evaluations is required. In addition, prior to each study session, all subjects must abstain from all psychoactive drugs for a period of 72 hours, alcohol for 24 hours, caffeine for 8 hours, food for two hours and use of tobacco products for 30 minutes. Finally, the subjects are required to provide a urine sample for illicit drug screening (ABUSCREEN™ urine test kit) before each study session.

Exclusion Criteria:

Subjects who meet the following criteria will be excluded from the study: women who are pregnant or lactating; women who are attempting to become pregnant or who are not using a medically approved method of contraception and are sexually active; subjects with a known hypersensitivity to stimulants or who have had an adverse reaction from previous stimulant abuse; the presence of an organic brain syndrome; a history of cardiac disease or pathologically abnormal ECG including conduction defects; a history of seizure disorders, severe cerebral trauma or stroke; administration of an investigational drug within 30 days of the start of the study; or a likelihood that the subject will require a medical treatment that may interfere with the CNS properties of the study drugs including their disposition.

Grouping of Subjects:

For adequate statistical power, the subjects are randomly divided into four groups with six subjects per group with each of the four groups being randomly assigned to one of four sequences. Four study sessions are held at weekly intervals in a living room setting to facilitate interaction between the subjects. In all four-study sessions, each subject is requested to take one oral capsule and to submit to an intravenous injection. At least one of these dosage forms will contain a placebo (either the oral placebo or the intravenous placebo). All injections are administered slowly over a period of two minutes (a stop watch is used to measure the administration period). The subjects may not interact when completing their self-rating evaluations.

Dosage Forms:

Intravenous dosage forms are prepared extemporaneously in a laminar airflow hood by dissolving pure 20 mg d-MPH hydrochloride or 20 mg pure l-MPH hydrochloride in sterile water for injection under aseptic conditions. The intravenous dosage forms are tested commercially for sterility and for absence of pyrogens. Sterile saline for injection is purchased commercially. The oral dosage forms are prepared extemporaneously by adding either 30 mg dextroamphetamine sulfate or 30 mg lactose to opaque capsules.

Ratings:

Before each session, it must be confirmed that each subject has adhered to the restrictions on psychoactive drugs, alcohol, caffeine, food and tobacco products as outlined in the Inclusion Criteria above. After administration of each treatment (i.e., the study drug or placebo), the subjects are asked to complete the following five ratings scales at hours 1, 2, 3, and 4 after dosing: (1) the Addiction Research Center Inventory, used to evaluate the subjective effects of the drug in relation to other well known drugs of abuse; (2) treatment identification, in which the subjects are asked to guess the identity of the treatment and evaluate how much they enjoy it; (3) feelings statements, in which the subjects are asked to rate how the drug makes them feel on a linear scale; (4) street value, in which the subjects are asked to estimate the cash value of the treatment, taking into consideration such negative feelings, such as dysphoria, for which they would not pay to repeat the experience; (5) profile of mood states, in which subjects are asked to choose from a list of 40 adjectives to describe how they feel at any given time.

Other Study Specifics:

A meal is provided during the second hour. All subjects are kept under close medical observation during the entire procedure and all medical events, serious and non-serious are reported to the clinical coordinator. At the end of each session, all subjects are monitored for side effects. A taxi service is provided to take each subject home after each session. Ten days after the completion of the study, each subject is to return for a post study physical examination and laboratory tests.

### EXAMPLE 2

### Plasma Concentration and Pharmacokinetic Data of d-threo and l-threo Methylphenidate After IV and IR Oral Administration of Racemic Methylphenidate

Eleven healthy male subjects were administered 10 mg of racemic methylphenidate (Ritalin®) by intravenous (IV) administration and 40 mg of racemic methylphenidate (Ritalin®) by immediate release (IR) oral administration. Mean plasma concentration versus time plots for d-MPH and l-MPH for each of the eleven subjects is shown in FIG. 1.

Table 1 sets forth the pharmacokinetic parameters for the d-threo and l-threo methylphenidate enantiomers in the plasma of the eleven subjects after IV administration of 10 mg of methylphenidate.

TABLE 1
PHARMACOKINETIC PARAMETERS OF d-threo AND l-threo METHYLPHENIDATE AFTER
INTRAVENOUS ADMINISTRATION OF 10 MG OF METHYLPHENIDATE

| | CL | | MRT | | Vdₛₛ | | AUC | | t_{½} | |
|---|---|---|---|---|---|---|---|---|---|---|
| | (L/kg · h⁻¹) | | (H) | | (L/kg) | | (ng/mL · h⁻¹) | | (h) | |
| Subject | d | l | d | l | d | l | d | l | d | l |
| 1 | 0.41 | 1.11 | 9.44 | 3.03 | 3.91 | 3.36 | 111.64 | 41.69 | 8.68 | 4.06 |
| 2 | 0.42 | 1.02 | 8.80 | 2.84 | 3.68 | 2.89 | 119.67 | 49.15 | 8.62 | 5.23 |
| 3 | 0.32 | 0.56 | 6.49 | 2.28 | 2.09 | 1.27 | 196.16 | 113.14 | 4.88 | 2.08 |
| 4 | 0.45 | 0.82 | 4.63 | 2.28 | 2.10 | 1.86 | 132.59 | 73.69 | 4.81 | 4.19 |
| 5 | 0.33 | 0.59 | 5.23 | 2.02 | 1.73 | 1.19 | 168.55 | 94.36 | 4.17 | 2.63 |
| 6 | 0.28 | 0.41 | 5.75 | 2.37 | 1.59 | 0.97 | 164.16 | 113.52 | 5.38 | 3.57 |
| 7 | 0.53 | 1.05 | 6.25 | 2.63 | 3.28 | 2.77 | 99.98 | 50.01 | 6.65 | 5.11 |
| 8 | 0.20 | 0.26 | 4.68 | 2.12 | 0.92 | 0.56 | 259.39 | 192.26 | 3.97 | 2.93 |
| 9 | 0.63 | 0.89 | 6.79 | 2.50 | 4.27 | 2.23 | 109.62 | 77.20 | 6.93 | 4.19 |
| 10 | 0.34 | 0.49 | 5.86 | 3.34 | 2.01 | 1.63 | 152.56 | 107.15 | 4.65 | 2.21 |
| 11 | 0.44 | 0.78 | 7.90 | 1.42 | 3.51 | 1.03 | 110.80 | 62.88 | 6.80 | 3.47 |
| mean | 0.40 | 0.73 | 6.53 | 2.44 | 2.65 | 1.80 | 147.74 | 88.64 | 5.96 | 3.61 |
| (Std dev) | (0.12) | (0.28) | (1.62) | (0.5) | (1.11) | (0.91) | (47.91) | (43.16) | (1.71) | (1.12) |

Paired t-tests (^{a}df = 10)

| | | | | | |
|---|---|---|---|---|---|
| t^{a} | −5.400 | 8.760 | 3.877 | 12.948 | 6.625 |
| α | <0.001 | <0.001 | <0.005 | <0.001 | <0.001 |

Table 2 sets for the pharmacokinetic parameters of d-threo and l-threo methylphenidate enantiomers in the plasma of the eleven subjects after oral administration of 40 mg of IR methylphenidate.

TABLE 2
PHARMACOKINETIC PARAMETERS OF d-threo AND l-threo
METHYLPHENIDATE AFTER ORAL ADMINISTRATION
OF 40 MG OF IMMEDIATE RELEASE METHYLPHENIDATE

| | Tmax | | Cmax | | AUC¹ | | AUC | | t_{½} | |
|---|---|---|---|---|---|---|---|---|---|---|
| | (h) | | (ng/mL) | | (ng/mL · h⁻¹) | | (ng/mL · h⁻¹) | | (h) | |
| Subject | d | l | d | l | d | l | d | l | d | l |
| 1 | 4.0 | 4.0 | 13.24 | 3.95 | 84.17 | 16.00 | 112.62 | 18.57 | 8.38 | 5.41 |
| 2 | 2.0 | 2.0 | 14.68 | 4.23 | 88.83 | 19.54 | 109.41 | 21.54 | 5.44 | 4.61 |
| 3 | 3.0 | 3.0 | 12.42 | 2.49 | 68.21 | 12.45 | 81.97 | 13.31 | 6.67 | 2.48 |
| 4 | 3.0 | 3.0 | 16.49 | 3.26 | 138.07 | 16.87 | 158.49 | 18.27 | 4.44 | 4.06 |
| 5 | 3.0 | 3.0 | 13.81 | 1.59 | 74.85 | 9.42 | 88.21 | 11.12 | 5.83 | 3.80 |
| 6 | 2.0 | 2.0 | 22.02 | 3.61 | 129.39 | 12.81 | 143.90 | 14.19 | 4.14 | 3.09 |
| 7 | 1.0 | 1.0 | 18.52 | 1.49 | 54.28 | 5.09 | 68.97 | 5.89 | 6.22 | 3.91 |
| 8 | 2.0 | 2.0 | 24.54 | 2.88 | 133.89 | 13.13 | 155.25 | 15.97 | 4.81 | 4.24 |
| 9 | 2.0 | 2.0 | 22.48 | 4.01 | 114.53 | 12.73 | 136.53 | 15.13 | 6.31 | 3.84 |
| 10 | 2.0 | 1.5 | 18.25 | 2.27 | 100.42 | 11.17 | 122.30 | 13.89 | 6.09 | 3.55 |
| 11 | 2.0 | 2.0 | 22.91 | 3.03 | 118.45 | 12.84 | 144.68 | 14.97 | 5.29 | 4.23 |
| mean | 2.4 | 2.2 | 18.12 | 2.98 | 1100.46 | 12.91 | 120.21 | 14.79 | 5.69 | 3.93 |
| (Std dev) | (0.8) | (0.6) | (4.3) | (0.9) | (28.5) | (3.8) | (30.7) | (4.1) | (1.1) | (0.8) |

Paired t-tests (^{a}df = 10)

| | | | | | |
|---|---|---|---|---|---|
| t^{a} | 1.242 | 11.626 | 10.754 | 12.222 | 5.210 |
| α | NS | <0.001 | <0.001 | <0.001 | <0.001 |

### EXAMPLE 3

### Preparation of “Abuse Free” Oral Methylphenidate Formulations

TABLET 1 (ABUSE FREE MPH
WITH 5% APOMORPHINE HYDROCHLORIDE):

| Component | Function | Amount per tablet |
|---|---|---|
| Racemic methylphenidate | Active agent | 4.00 mg |
| Apomorphine hydrochloride | Active agent | 3.00 mg |
| Dicalcium phosphate dihydrate | Diluent | 24.10 mg |
| Microcrystalline cellulose | Diluent | 27.10 mg |
| Sodium starch glycolate | Disintegrant | 1.2 mg |
| Magnesium Stearate | Lubricant | 0.6 mg |

TABLET 2
(ABUSE-FREE MPH WITH 0.5% WT. CAPSAICIN):

| Component | Function | Amount per tablet |
|---|---|---|
| Racemic methylphenidate | Active agent | 4.00 mg |
| Capsaicin hydrochloride | Active agent | 0.3 mg |
| Dicalcium phosphate dihydrate | Diluent | 26.80 mg |
| Microcrystalline cellulose | Diluent | 27.10 mg |
| Sodium starch glycolate | Disintegrant | 1.2 mg |
| Magnesium Stearate | Lubricant | 0.6 mg |

### EXAMPLE 4

### Pulsatile Delivery System for the Oral Administration of Enantiomerically Pure d-threo Methylphenidate

A pulsatile release dosage form for administration of enantiomerically pure d-threo methylphenidate is prepared by (1) formulating three individual compressed tablets, each having a different release profile, followed by (2) encapsulating the three tablets in a gelatin capsule and then closing and sealing the capsule. The third tablet contains half the amount of active agent found in the first and second tablets. The components of the three tablets are as follows:

TABLET 1 (IMMEDIATE RELEASE):

| Component | Function | Amount per tablet |
|---|---|---|
| d-threo methylphenidate | Active agent | 2.00 mg |
| Dicalcium phosphate dihydrate | Diluent | 28.10 mg |
| Microcrystalline cellulose | Diluent | 28.10 mg |
| Sodium starch glycolate | Disintegrant | 1.2 mg |
| Magnesium Stearate | Lubricant | 0.6 mg |

TABLET 2 (RELEASE DELAYED 3-5
HOURS FOLLOWING ADMINISTRATION):

| Component | Function | Weight |
|---|---|---|
| Tablet 1 | “Core” containing the active agent | 60.0 mg |
| Eudragit RS30D | Delayed release coating material | 4.76 mg |
| Talc | Coating component | 3.3 mg |
| Triethyl citrate | Coating component | 0.95 mg |

TABLET 3A:

| Component | Function | Amount per tablet |
|---|---|---|
| d-threo methylphenidate | Active agent | 1.00 mg |
| Dicalcium phosphate dihydrate | Diluent | 14.05 mg |
| Microcrystalline cellulose | Diluent | 44.05 mg |
| Sodium starch glycolate | Disintegrant | 0.6 mg |
| Magnesium Stearate | Lubricant | 0.3 mg |

TABLET 3B:

| Component | Function | Weight |
|---|---|---|
| Tablet 3A | “Core” containing the active agent | 60.0 mg |
| Eudragit RS30D | Delayed release coating material | 6.34 mg |
| Talc | Coating component | 4.4 mg |
| Triethyl citrate | Coating component | 1.27 mg |

The tablets are prepared by wet granulation of an aqueous solution of the individual components as may be done using a fluid-bed granulator, or are prepared by direct compression of the admixture of components. Tablet 1 is an immediate release dosage form, releasing the active agent within 1-2 hours following administration. Tablets 2 and 3, after this initial preparation, are coated with the delayed release coating material such as may be carried out using conventional coating techniques such as spray-coating or the like. As will be appreciated by those skilled in the art, the specific components listed in the above tables may be replaced with other functionally equivalent components, e.g., diluents, binders, lubricants, fillers, coatings, and the like.

Oral administration of the capsule to a patient will result in a release profile having three pulses, with initial release of the methylphenidate from the first tablet being substantially immediate, release of the methylphenidate from the second tablet occurring 3-5 hours following administration, and release of the methylphenidate from the third tablet occurring 7-9 hours following administration.

Capsules may be prepared with apomorphine and capsaicin in addition to the methylphenidate as described in Example 3.

### EXAMPLE 5

### Pulsatile Delivery System for Oral Administration of a 99% d-threo/1% l-threo Methylphenidate Formulation

A pulsatile release dosage form for administration of methylphenidate is prepared by (1) formulating three individual compressed tablets as in Example 3, but substituting 4.5 mg d-threo methylphenidate and 0.5 mg l-threo methylphenidate for the 1.70 mg d-threo methylphenidate in each tablet, followed by (2) encapsulating the three tablets into a capsule as described in Example 4. The third tablet contains half the amount of active agent found in the first and second tablets. The components of the three tablets are as follows

TABLET 1 (IMMEDIATE RELEASE):

| Component | Function | Amount per tablet |
|---|---|---|
| d-threo methylphenidate | Active agent | 3.96 mg |
| l-threo methylphenidate | Active agent | 0.04 mg |
| Dicalcium phosphate dihydrate | Diluent | 27.10 mg |
| Microcrystalline cellulose | Diluent | 27.10 mg |
| Sodium starch glycolate | Disintegrant | 1.2 mg |
| Magnesium Stearate | Lubricant | 0.6 mg |

TABLET 2 (RELEASE DELAYED 3-5
HOURS FOLLOWING ADMINISTRATION):

| Component | Function | Weight |
|---|---|---|
| Tablet 1 | “Core” containing d-threo and l- | 60.0 mg |
| | threo methylphenidate | |
| Eudragit RS30D | Delayed release coating material | 4.76 mg |
| Talc | Coating component | 3.3 mg |
| Triethyl citrate | Coating component | 0.95 mg |

TABLET 3 (RELEASE DELAYED 7-9
HOURS FOLLOWING ADMINISTRATION):
TABLET 3A:

| Component | Function | Amount per tablet |
|---|---|---|
| d-threo methylphenidate | Active agent | 1.98 mg |
| l-threo methylphenidate | Active agent | 0.02 mg |
| Dicalcium phosphate dihydrate | Diluent | 13.55 mg |
| Microcrystalline cellulose | Diluent | 43.55 mg |
| Sodium starch glycolate | Disintegrant | 0.6 mg |
| Magnesium Stearate | Lubricant | 0.3 mg |

TABLET 3B:

| Component | Function | Weight |
|---|---|---|
| Tablet 3A | “Core” containing d-threo and l-threo | 60.0 mg |
| | methylphenidate | |
| Eudragit RS30D | Delayed release coating material | 6.34 mg |
| Talc | Coating component | 4.4 mg |
| Triethyl citrate | Coating component | 1.27 mg |

Oral administration of the capsule to a patient will result in a release profile having three pulses, with initial release of the methylphenidate from the first tablet being substantially immediate, release of the methylphenidate from the second tablet occurring 3-5 hours following administration, and release of the methylphenidate from the third tablet occurring 7-9 hours following administration.

Capsules may be prepared with apomorphine and capsaicin in addition to the methylphenidate as described in Example 3.

### EXAMPLE 6

### Combined Pharmacokinetic/Pharmacodynamic Studies of the Effects of Different Dose Regiments of Methylphenidate on Activity and Attention

Prescreening Process:

Combined pharmacokinetic/pharmacodynamic studies of methylphenidate using different dosage regiments were designed to determine if regimens that produced stable or rising methylphenidate levels had benefits, or if fluctuating levels were necessary to prevent rapid development of tolerance (tachyphylaxis). The experimental subjects were a group of seventy-three males with ADHD recruited from the general population through a newspaper advertisement. Each subject was between the ages of 9 to 12, was objectively hyperactive, and responded positively to methylphenidate. Hyperactivity levels of each boy were determined through OPTAX™ measures, a clinical tool that provides high-resolution assessment of movement patterns during performance of a novel, monotonous, yet challenging cognitive vigilance task. Through the OPTAX™ measures, it is possible to identify differences between drug regimens based solely on dosage schedule, while holding total daily dose constant. Of the 73 original boys, thirteen were excluded based on lack of symptoms by parent interview or on lack of objective hyperactivity greater than 25% over controls on OPTAX™ measures. For the remaining 60 boys, a single probe dose of MPH at 0.4 mg/kg body weight was administered to assess response. Of these 60 boys, twelve were excluded based upon lack of improvement post-MPH on OPTAX™, drop-out between the screening visit and the test day, and anxiety or refusal during insertion of the intravenous on the test day. Forty-eight boys with a mean age of 10.6±1.1 yr completed the entire protocol.

Procedure:

The 48 boys were studied in groups of 4 to 6 on Saturdays from 7:00 a.m. to 7:00 p.m., randomized to one of the four dosing schedules in a hospital setting. The hospital pharmacy prepared individual dose capsules labeled only with the child's name and time of administration. The boys arrived at the lab medication-free before breakfast. An indwelling intravenous (IV) catheter was inserted in their nondominant arm through which twelve blood samples were drawn at hour intervals throughout the day. Baseline blood levels and OPTAX™ measures were obtained before the first dosing. Subjects were clustered in two groups for parallel schedules of breakfast, lunch, and snacks, approximately one half hour apart, in order to maintain proportionally equivalent timing of food and drug dose for each child. The boys followed a strict individual hourly schedule of blood drawing via IV site, followed by five minutes of OPTAX™ testing, then medication administration. The boys received the contents of a capsule each hour, either active drug or lactose powder dissolved in fruit juice and consumed immediately. Blood levels of six boys were sent but not processed by the lab and blood samples of two boys were not collected. For the final analysis, 40 boys had complete blood data as follows: Continuous Schedule 1 (CD1) (n=9), Continuous Schedule 2 (CD2) (n=11), Pulsatile Schedule 1 (PD1) (n=10), and Pulsatile Schedule 2 (PD2) (n=10).

Drug Administration Schedules:

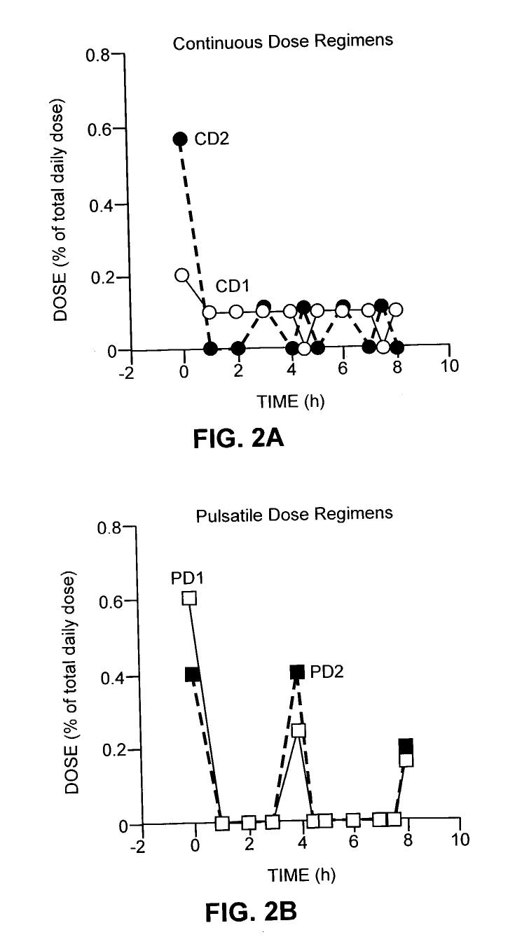

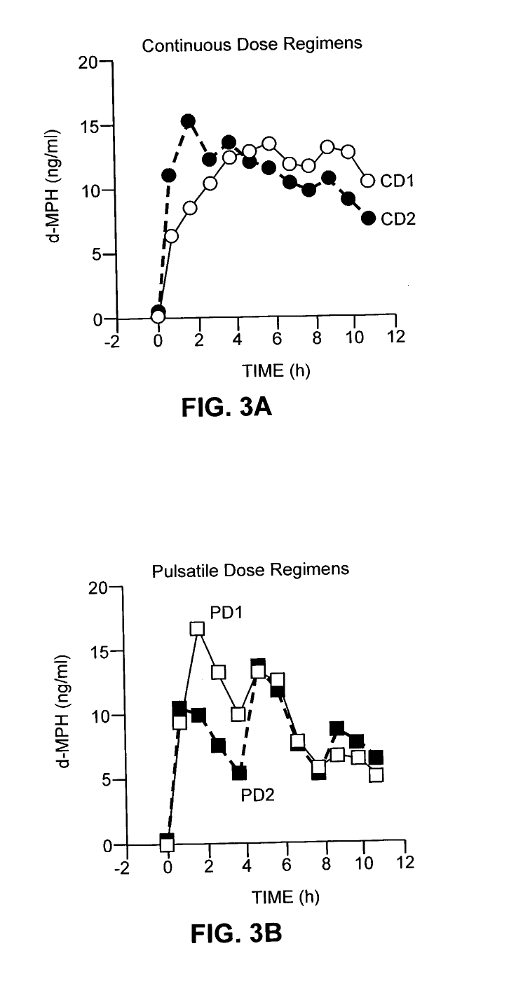

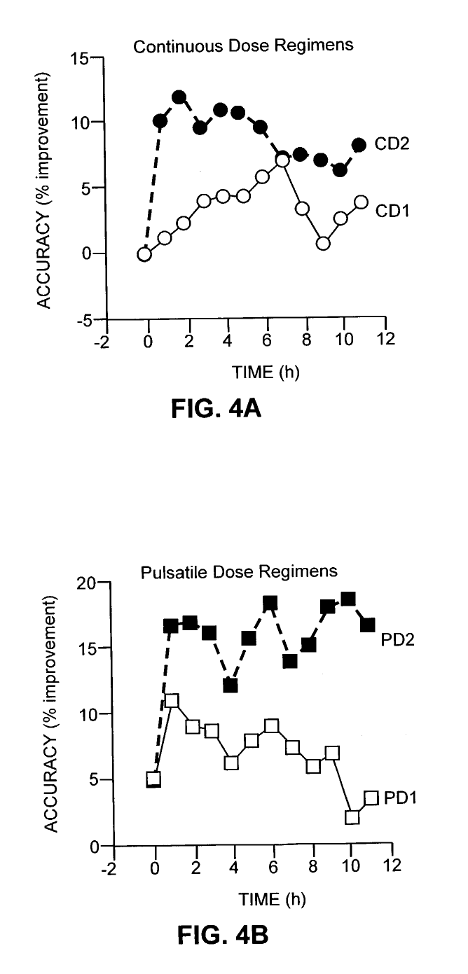

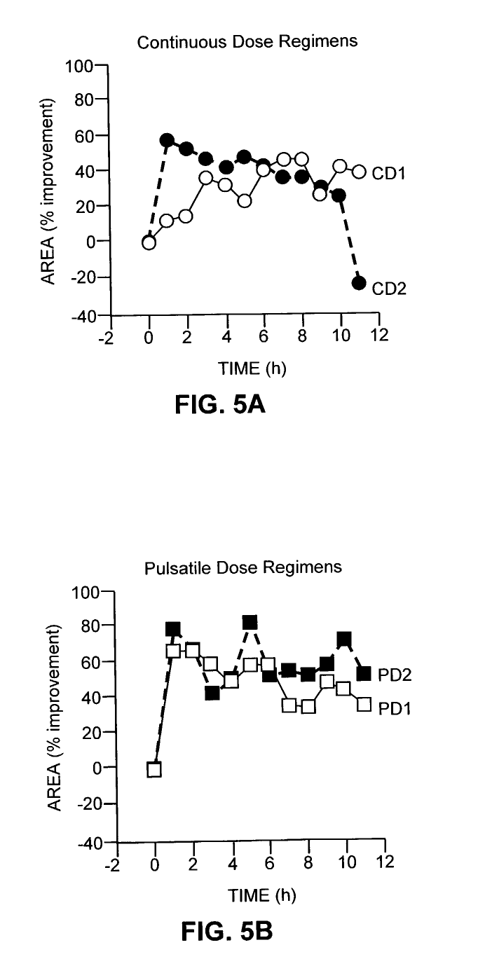

The double blind parallel group study evaluated the efficacy of methylphenidate for the treatment of ADHD when given in a near continuous amount throughout the day and in pulsatile or fluctuating doses throughout the day. The total daily dose of 1 mg/kg/day methylphenidate was administered in accordance with four administration paradigms in divided doses (proposed n=12 subjects per group). In CD1, subjects received 0.2 mg/kg at time zero followed by eight doses of 0.1 mg/kg at subsequent hourly intervals. In CD2, subjects received 0.566 mg/kg at time zero followed by 0.11 mg/kg 180 minutes later, 0.111 at 270 minutes later, 0.111 at 360 minutes later, and 0.11 at 450 minutes post start. A graphic illustration of the distribution dose by time for the near continuous dosing schedules is provided in FIG. 2A. The pharmacokinetic profile of d-MPH levels following administration of methylphenidate for the near continuous dosing schedules is provided in FIG. 3A. In PD1, subjects received 0.6 mg/kg at time zero, 0.24 mg/kg at 240 minutes later, and 0.16 mg/kg at 480 minutes post start. In PD2, subjects received 0.4 mg/kg at time zero, 0.4 mg/kg 240 minutes later and 0.2 mg/kg at 480 minutes post start. A graphic illustration of the distribution dose by time for the pulsatile dosing schedules is provided in FIG. 2B. The pharmacokinetic profile of d-MPH levels following administration of methylphenidate for the pulsatile dosing schedules is provided in FIG. 3B. FIGS. 4A and 4B show the percent improvement in attention of the subjects in each of the four dosing groups over the over the course of the twelve hour day. FIGS. 5A and 5B show the percent improvement in activity of the subjects in each of the four dosing groups over the course of the twelve-hour day. Pulsatile dosing schedule 2 (PD2) showed the most consistent improvement in both attention (FIG. 4B) and activity (FIG. 5B).

The foregoing experiment was conducted with commercially available racemic methylphenidate and not with pure d-threo or pure l-threo methylphenidate due to the ethical and practical consideration involved in a study where the subjects are small children; however, the foregoing results, when combined with the results of experiment 2, indicate that the results reported herein for commercially available racemic methylphenidate would be similar or superior if conducted with pure d-threo methylphenidate or with methylphenidate substantially free of the l-threo enantiomer.

1. A method for reducing the likelihood that a methylphenidate product will be abused, comprising incorporating one of an emesis-inducing agent and a topical analgesic into the methylphenidate product, wherein the emesis-inducing agent and the topical analgesic are inert in oral dosage forms and active only when the methylphenidate is administered non-medically.

2. The method of claim 1, wherein the methylphenidate product is racemic methylphenidate (MPH), pure d-threo MPH (d-MPH), or MPH substantially free of l-threo MPH (l-MPH).

3. The method of claim 1, wherein the emesis-inducing agent is apomorphine.

4. The method of claim 1, wherein the topical analgesic is capsaicin.

5. A method for treating a methylphenidate-responsive condition in a patient with methylphenidate while minimizing the likelihood that the patient will abuse or become addicted to the methylphenidate, the method comprising:
(a) determining whether the patient has an elevated risk of abusing or becoming addicted to the methylphenidate and if the determination is that the patient does have an elevated risk of abusing or becoming addicted to the methylphenidate, then,
(b) treating the methylphenidate-responsive condition by orally administering to the patient a methylphenidate product that includes one of an emesis-inducing agent and a topical analgesic, wherein the emesis-inducing agent and the topical analgesic are inert in oral dosage forms and active only when the methylphenidate is administered non-medically.

6. The method of claim 5, wherein the methylphenidate product is racemic methylphenidate (MPH), pure d-threo MPH (d-MPH), or MPH substantially free of l-threo MPH (l-MPH).

7. The method of claim 5, wherein the emesis-inducing agent is apomorphine.

8. The method of claim 5, wherein the topical analgesic is capsaicin.

9. The method of claim 5, wherein the patient is at least 18 years of age.

10. The method of claim 5, wherein the patient is 9 to 17 years old.

11. The method of claim 5, wherein the patient is under 9 years of age.

12. The method of claims 9 or 10, wherein (a) includes evaluating the patient's psychological profile to identify factors contributing to an elevated risk of drug abuse or addiction.

13. The method of claims 9 or 10, wherein (a) includes evaluating the patient's history of drug abuse or addiction.

14. The method of claims 9, 10, or 11, wherein (a) includes evaluating the patient's family history of drug abuse or addiction.

15. The method of claim 9, wherein the elevated risk comprises a substantially greater likelihood that the patient will abuse or become addicted to methylphenidate relative to the likelihood that an average patient in a population pool of patients at least 18 years of age will abuse or become addicted to methylphenidate.

16. The method of claim 15, wherein the patients in the population pool exhibit a methylphenidate-responsive condition.

17. The method of claim 10, wherein the elevated risk comprises a substantially greater likelihood that the patient will abuse or become addicted to methylphenidate relative to the likelihood that an average patient in a population pool of patients of ages 9 to 17 years of age will abuse or become addicted to methylphenidate.

18. The method of claim 17, wherein the patients in the population pool exhibit a methylphenidate-responsive condition.

19. The method of claim 5, wherein the methylphenidate-responsive condition is selected from the group consisting of attention deficit disorder (ADD), attention deficit hyperactivity disorder (ADHD), narcolepsy, chronic fatigue syndrome, acute depression, bulimia, cognitive decline associated with Acquired Immunodeficiency Syndrome (AIDS) or AIDS-related conditions, and a depressed mood in a terminally ill patient.

20. A method for treating a methylphenidate-responsive condition in a patient with methylphenidate while minimizing the likelihood that the patient will abuse or become addicted to the methylphenidate, the method comprising:
(a) determining whether the patient has an elevated risk of abusing or becoming addicted to the methylphenidate and if the determination is that the patient does have an elevated risk of abusing or becoming addicted to the methylphenidate, then,
(b) treating the methylphenidate-responsive condition by orally administering to the patient, once daily, a pulsatile release dosage form comprised of an immediate release dosage unit and a delayed release dosage unit, each said dosage unit containing a methylphenidate product, wherein following oral administration of the dosage form, a first dose of the methylphenidate product is released substantially immediately from the immediate release dosage unit, followed by a time interval during which substantially no drug is released from the dosage form, and after which time interval a second dose of the methylphenidate product is released from the delayed release dosage unit, and wherein the methylphenidate product includes one of an emesis-inducing agent and a topical analgesic, wherein the emesis-inducing agent and the topical analgesic are inert in oral dosage forms and active only when the methylphenidate is administered non-medically.

21. The method of claim 20, wherein the methylphenidate product is racemic methylphenidate (MPH), pure d-threo MPH (d-MPH), or MPH substantially free of l-threo MPH (l-MPH).

22. The method of claim 20, wherein the emesis-inducing agent is apomorphine.

23. The method of claim 20, wherein the topical analgesic is capsaicin

24. The method of claim 20, wherein the patient is at least 18 years of age.

25. The method of claim 20, wherein the patient is 9 to 17 years old.

26. The method of claim 20, wherein the patient is under 9 years of age.

27. The method of claims 24 or 25, wherein (a) includes evaluating the patient's psychological profile to identify factors contributing to an elevated risk of drug abuse or addiction.

28. The method of claims 24 or 25, wherein (a) includes evaluating the patient's history of drug abuse or addiction.

29. The method of claims 24, 25, or 26, wherein (a) includes evaluating the patient's family history of drug abuse or addiction.

30. The method of claim 24, wherein the elevated risk comprises a substantially greater likelihood that the patient will abuse or become addicted to methylphenidate relative to the likelihood that an average patient in a population pool of patients at least 18 years of age will abuse or become addicted to methylphenidate.

31. The method of claim 30, wherein the patients in the population pool exhibit a methylphenidate-responsive condition.

32. The method of claim 25, wherein the elevated risk comprises a substantially greater likelihood that the patient will abuse or become addicted to methylphenidate relative to the likelihood that an average patient in a population pool of patients of ages 9 to 17 years of age will abuse or become addicted to methylphenidate.

33. The method of claim 32, wherein the patients in the population pool exhibit a methylphenidate-responsive condition.

34. The method of claim 20, wherein the methylphenidate-responsive condition is is selected from the group consisting of attention deficit disorder (ADD), attention deficit hyperactivity disorder (ADHD), narcolepsy, chronic fatigue syndrome, acute depression, bulimia, cognitive decline associated with Acquired Immunodeficiency Syndrome (AIDS) or AIDS-related conditions, and a depressed mood in a terminally ill patient.

35. The method of claim 20, wherein the dosage form is a capsule and the immediate release and delayed release dosage units are tablets contained therein.

36. The method of claim 20, wherein the dosage form is a tablet, and the immediate release and delayed release dosage units are integral and discrete segments thereof.

37. The method of claim 20, wherein the first and second doses are approximately equal.

38. The method of claim 20, wherein at least one of the first and second dosage units further comprise an additional drug.

39. The method of claim 38, wherein the additional drug is selected from the group consisting of a stimulant, a methamphetamine, d-methamphetamine, amphetamine, d-amphetamine, pemoline, aspirin, acetaminophen, and psuedoephedrine.

40. The method of claim 20, wherein the means for delaying release comprises a coating of a delayed release membrane material.

41. The method of claim 40, wherein the delayed release membrane material is comprised of a bioerodible, hydrolyzable and/or gradually water-soluble polymer.

42. The method of claim 41, wherein the delayed release membrane material is an acrylic polymer or copolymer.

43. The method of claim 42, wherein the delayed release membrane material is a copolymer of acrylic acid, methacrylic acid, methyl acrylate, ethyl acrylate, methyl methacrylate, ethyl methacrylate, and/or derivatives thereof.

44. The method of claim 42, wherein the delayed release membrane material is a terpolymer of ethyl acrylate, methyl methacrylate and trimethylammonioethyl methacrylate chloride.

45. A method for treating a methylphenidate-responsive condition in a patient with methylphenidate while minimizing the likelihood that the patient will abuse or become addicted to the methylphenidate, the method comprising:
(a) determining whether the patient has an elevated risk of abusing or becoming addicted to the methylphenidate and if the determination is that the patient does have an elevated risk of abusing or becoming addicted to the methylphenidate, then,
(b) treating the methylphenidate-responsive condition by orally administering to the patient, once daily, a pulsatile release dosage form comprised of an immediate release dosage unit and a delayed release dosage unit, each said dosage unit containing a methylphenidate product comprised of d-threo methylphenidate (d-MPH) and substantially no l-threo methylphenidate (l-MPH), wherein following oral administration of the dosage form, a first dose of d-MPH is released substantially immediately from the immediate release dosage unit, followed by a time interval during which substantially no drug is released from the dosage form, and after which time interval a second dose of d-MPH is released from the delayed release dosage unit, and wherein the methylphenidate product includes one of an emesis-inducing agent and a topical analgesic, wherein the emesis-inducing agent and the topical analgesic are inert in oral dosage forms and active only when the methylphenidate is administered non-medically.

46. The method of claim 45, wherein the emesis-inducing agent is apomorphine.

47. The method of claim 45, wherein the topical analgesic is capsaicin.

48. The method of claim 45, wherein the patient is at least 18 years of age.

49. The method of claim 45, wherein the patient is 9 to 17 years old.

50. The method of claim 45, wherein the patient is under 9 years of age.

51. The method of claims 48 or 49, wherein (a) includes evaluating the patient's psychological profile to identify factors contributing to an elevated risk of drug abuse or addiction.

52. The method of claims 48 or 49, wherein (a) includes evaluating the patient's history of drug abuse or addiction.

53. The method of claims 48, 49, or 50, wherein (a) includes evaluating the patient's family history of drug abuse or addiction.

54. The method of claim 48, wherein the elevated risk comprises a substantially greater likelihood that the patient will abuse or become addicted to methylphenidate relative to the likelihood that an average patient in a population pool of patients at least 18 years of age will abuse or become addicted to methylphenidate.

55. The method of claim 54, wherein the patients in the population pool exhibit a methylphenidate-responsive condition.

56. The method of claim 49, wherein the elevated risk comprises a substantially greater likelihood that the patient will abuse or become addicted to methylphenidate relative to the likelihood that an average patient in a population pool of patients of ages 9 to 17 years of age will abuse or become addicted to methylphenidate.

57. The method of claim 56, wherein the patients in the population pool exhibit a methylphenidate-responsive condition.

58. The method of claims 56, wherein the methylphenidate-responsive condition is is selected from the group consisting of attention deficit disorder (ADD), attention deficit hyperactivity disorder (ADHD), narcolepsy, chronic fatigue syndrome, acute depression, bulimia, cognitive decline associated with Acquired Immunodeficiency Syndrome (AIDS) or AIDS-related conditions, and a depressed mood in a terminally ill patient.

59. The method of claim 45, wherein the ratio of d-MPH:l-MPH is at least 85:15.

60. The method of claim 45, wherein the ratio d-MPH:l-MPH is at least 90:10.

61. The method of claim 45, wherein the ratio of d-MPH:l-MPH is at least 95:5.

62. The method of claim 45, wherein the ratio of d-MPH:l-MPH is at least 99:1.

63. The method of claim 45, wherein the first dose of d-MPH is released within 2 hours of oral administration of the dosage form.

64. The method of claim 63, wherein the first dose of d-MPH is released within 1 hour of oral administration of the dosage form.

65. The method of claim 64, wherein the second dosage unit additionally comprises a means for delaying release of the second dose of d-MPH until approximately 3 to 5 hours following oral administration of the dosage form.

66. The method of claim 65, wherein the second dosage unit additionally comprises a means for delaying release of the second dose of d-MPH until approximately 3 to 5 hours following oral administration of the dosage form.

67. The method of claims 45, wherein the dosage form is a capsule and the immediate release and delayed release dosage units are tablets contained therein.

68. The method of claim 67, wherein the dosage form is a capsule, the immediate release dosage unit is comprised of a plurality of beads or particles together containing the first dose of d-MPH, and the delayed release dosage unit is comprised of a plurality of beads or particles together containing the second dose of d-MPH.

69. The method of claim 45, wherein the dosage form is a tablet, and the immediate release and delayed-release dosage units are integral and discrete segments thereof.

70. The method of claim 69, wherein the total d-MPH in the dosage form is in the range of approximately 1 mg to 100 mg.

71. The method of claim 70, wherein the total d-MPH in the dosage form is in the range of approximately 2 mg to 50 mg.

72. The method of claim 71, wherein the first dose of d-MPH and the second dose of d-MPH are each in the range of approximately 0.5 mg to 20 mg.

73. The method of claim 45, wherein the first and second doses are approximately equal.

74. The method of claim 45, wherein at least one of the first and second dosage units further comprises an additional drug.

75. The method of claim 45, wherein the additional drug is selected from the group consisting of a stimulant, a methamphetamine, d-methamphetamine, amphetamine, d-amphetamine, pemoline, aspirin, acetaminophen, and psuedoephedrine.

76. The method of claims 45, wherein the means for delaying release comprises a coating of a delayed release membrane material.

77. The method of claim 76, wherein the delayed release membrane material is comprised of a bioerodible, hydrolyzable and/or gradually water-soluble polymer.

78. The method of claim 77, wherein the delayed release membrane material is an acrylic resin.

79. The method of claim 78, wherein the delayed release membrane material is a copolymer of acrylic acid, methacrylic acid, methyl acrylate, ethyl acrylate, methyl methacrylate, ethyl methacrylate, and/or derivatives thereof.

80. The method of claim 78, wherein the delayed release membrane material is a terpolymer of ethyl acrylate, methyl methacrylate and trimethylammonioethyl methacrylate chloride.

81. The method of claim 45, wherein the dosage form further comprises a third dosage unit comprising a third dose of d-threo methylphenidate (d-MPH).

82. The method of claim 81, wherein the third dose of d-MPH is released after an additional time interval following release of the second dose of d-MPH.

83. The method of claim 82, wherein the third dosage unit additionally comprises a means for delaying release of the third dose of d-MPH until approximately 7 to 9 hours following oral administration of the dosage form.

84. The method of claim 81, wherein the third dose of d-MPH is approximately half that of the first dose of d-MPH.

85. The method of claim 81, wherein the total d-MPH in the dosage form is in the range of approximately 1 mg to 100 mg.

86. The method of claim 85, wherein the total d-MPH in the dosage form is in the range of approximately 2 mg to 50 mg.

87. The method of claim 81, wherein the third dosage unit releases the third dose of d-MPH in the colon.
